(19) United States
(12) Patent Application Publication
(10) Pub. No.: US 2013/0104883 A1
(43) Pub. Date: May 2, 2013

(54) CONTINUOUS POSITIVE AIRWAY PRESSURE (CPAP) APPARAUTS WITH ORIENTATION SENSOR

(71) Applicant: Michael G. Lalonde, Alpharetta, GA (US)

(72) Inventor: Michael G. Lalonde, Alpharetta, GA (US)

(73) Assignee: DESHUM MEDICAL, LLC, Cambridge, MA (US)

(21) Appl. No.: 13/452,823

(22) Filed: Apr. 20, 2012

Related U.S. Application Data
(63) Continuation-in-part of application No. PCT/US2010/053370, filed on Oct. 20, 2010.
(60) Provisional application No. 61/253,500, filed on Oct. 20, 2009.
(60) Provisional application No. 61/288,290, filed on Dec. 19, 2009.
(60) Provisional application No. 61/301,151, filed on Feb. 3, 2010.
(60) Provisional application No. 61/560,271, filed on Nov. 15, 2011.

(51) Int. Cl. A61M 16/00; A61M 16/10; A61M 16/06

(52) U.S. Cl. CPC A61M 16/0057; A61M 16/06; A61M 16/1045
USPC 128/201.13; 128/204.21; 128/204.18

(57) ABSTRACT

A system and method for delaying the start of the continuous positive air pressure therein making it easier for a user to fall asleep. The system delivers pressurized gas to the airway of a patient. The system has a gas flow generator for providing a flow of gas and a mask for delivery of gas flow to an airway of a patient. The mask has an exhaust port being continuously open and having suitable flow resistance for maintaining a pressure in the cavity. The mask has a breathing port adaptable to open when there is no flow of pressurized air for allowing free breathing by the user. A hose extends between the gas flow generator and the mask for providing a flow of gas. The system has a mechanism for turning the flow of gas on at a time distinct from turning on the apparatus.

### CROSS-REFERENCE TO RELATED APPLICATIONS

This application is a continuation-in-part of PCT Application PCT/US2010/053370 filed on Oct. 20, 2010 which claims the benefit of U.S. Patent Application 61/253,500 filed on Oct. 20, 2009, U.S. Patent Application 61/288,290 filed on Dec. 19, 2009, and U.S. Patent Application 61/301,151 filed on Feb. 3, 2010 and this application claims the benefit of U.S. Patent Application 61/560,271 filed on Nov. 15, 2011, which are incorporated herein by reference.

### FIELD OF THE INVENTION

The present invention relates to a continuous positive airway pressure (CPAP) machine and more particularly to a CPAP machine that is activated based on the condition of the user and can be placed in various design forms.

### BACKGROUND OF THE INVENTION

Sleep apnea syndrome afflicts an estimated 1% to 5% of the general population and is due to episodic upper airway obstruction during sleep. Those afflicted with sleep apnea experience sleep fragmentation and intermittent, complete, or nearly complete cessation of ventilation during sleep with potentially severe degrees of oxyhemoglobin desaturation.

Although details of the pathogenesis of upper airway obstruction in sleep apnea patients have not been fully defined, it is generally accepted that the mechanism includes either anatomic or functional abnormalities of the upper airway which result in increased air flow resistance. Such abnormalities may include narrowing of the upper airway due to suction forces evolved during inspiration, the effect of gravity pulling the tongue back to oppose the pharyngeal wall, and/or insufficient muscle tone in the upper airway dilator muscles. It has also been hypothesized that a mechanism responsible for the known association between obesity and sleep apnea is excessive soft tissue in the anterior and lateral neck which applies sufficient pressure on internal structures to narrow the airway.

Recent work in the treatment of sleep apnea has included the use of continuous positive airway pressure (CPAP) to maintain the airway of the patient in a continuously open state during sleep. Unfortunately, the statistics on CPAP non-compliance are startling. There are numerous reasons for non-compliance including the discomfort of exhaling against a positive air pressure.

### SUMMARY OF THE INVENTION

It has been recognized that conventional CPAP (continuous positive airway pressure) machines to treat apnea provide a positive pressure to the user when the unit is turned on. The user is required to exhale, competing with the positive pressure from a flow generator. This competition against the CPAP machine is uncomfortable and not typical which results in difficulty falling asleep. It has been recognized that a CPAP system with a small blower or flow generator unit that can be placed at various locations including on the chest, in a pouch that can be placed on the chest, on the bed or other location, or the flow generator can be placed in other locations such as a docking station allows the user the ability to be more comfortable. In certain embodiments, the flow generator is integral with the mask.

In an embodiment of an apparatus for delivering pressurized gas to the airway of a patient, the apparatus includes a gas flow generator for providing a flow of gas, a mask for delivery of gas flow to an airway of a patient, and a connector between the gas flow generator and the mask for providing a flow of gas. The apparatus has a mechanism for turning the flow of the pressurized gas to the mask on and off; the turning on and off of the pressurized gas may be distinct from turning on the apparatus.

In an embodiment, the apparatus has an orientation sensor wherein the orientation sensor can influence when the flow of pressurized air is turned on and off.

In an embodiment of a mask for delivery of gas flow to an airway of a patient, the mask has a shell including a rim defining a cavity adapted for interface with a user's nose and mouth. The shell has a connection aperture. The mask has a mask connector which interfaces with the connection aperture of the shell. The connector defines a conduit for the flow of pressurized air from a flow generator. The mask has an exhaust port being continuously open and having suitable flow resistance for maintaining a pressure in the cavity. The mask has a breathing port adaptable to open when there is no flow of pressurized air for allowing free breathing by the user.

In an embodiment, the mask has a heat moisture exchange (HME) carried by the mask connector. The HME collects moisture on exhaling and provides moisture to the air on inhaling.

In an embodiment, the mask has a port defining a confined space. The port is adapted to connect a sensor carried on the flow generator. A flexible membrane, a button, covers the port and is adapted to change the volume of the confined space therein influencing the sensor.

In an embodiment, the mask has a second port adapted to connect to a sensor carried by the flow generator unit for controlling the air flow from the flow generator unit to the mask.

These aspects of the invention are not meant to be exclusive and other features, aspects, and advantages of the present invention will be readily apparent to those of ordinary skill in the art when read in conjunction with the following description, appended claims, and accompanying drawings.

### BRIEF DESCRIPTION OF THE DRAWINGS

The foregoing and other objects, features, and advantages of the invention will be apparent from the following description of particular embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a schematic of a CPAP system with sleep activity control system;

FIG. 2A is a sectional view of a portion of the mask;

FIG. 2B is a perspective view of the hose and the mask connector;

FIG. 3 is a top view of a flow generator unit;

FIG. 4 is an isometric view of the flow generator unit with a portion removed;

FIG. 5 is a sectional view of the flow generator unit taken along line 5-5 of FIG. 3;

FIG. 6A is an isometric view of a hose;

FIG. 6B is a side view of the hose;

FIG. 6C is an exploded view of the hose with the two components of the hose connectors separated;

FIG. 7 is a side view of the system on a user;

FIG. 8 is a schematic of an orientation sensor on a printed circuit board;

FIG. 9 is a perspective view of an alternative embodiment of the system;

FIG. 10 is a side view of a user wearing an alternative embodiment of the system;

FIG. 11 is a perspective view of the docking station;

FIG. 12A is a top view of the docking station of FIG. 11;

FIG. 12B is a front view of the docking station of FIG. 11;

FIG. 12C is a side view of the docking station;

FIG. 13 is a perspective view of the docking station with the upper clam shell hinged upward;

FIG. 14A is a top view of an alternative docking station;

FIG. 14B is the side view of the alternative docking station of FIG. 14A;

FIG. 15 is a perspective view of a pouch adapted for receiving the blower unit;

FIG. 16 is a top view of a pouch of FIG. 15;

FIG. 17 is a perspective view of an alternative pouch;

FIG. 18 is a sectional view of the pouch taken along the line 18-18 in FIG. 17;

FIG. 19 is a top view of the pouch showing internal components of a pair of batteries and a flow generator;

FIG. 20 is a sectional top view of the pouch;

FIG. 21 is a schematic of communications between the blower unit of the system and other devices;

FIG. 22 is a screen shot of an example of a clinical dashboard; and

FIG. 23 is a screen shot of a second opinion website.

### DETAILED DESCRIPTION OF THE INVENTION

A system and method for delivering pressurized gas to the airway of a patient, the system has a gas flow generator for providing a flow of gas and a mask for the delivery of the gas flow to an airway of a patient. The mask has a shell including a rim defining a cavity adapted for interface with a user's nose and mouth. The shell has a connection aperture. The mask has a mask connector interfacing with the connection aperture of the shell. The connector defines a conduit for the flow of pressurized air from the flow generator. The mask has an exhaust port being continuously open and having suitable flow resistance for maintaining a pressure in the cavity. The mask has a breathing port adaptable to open when there is no flow of pressurized air to allow for free breathing by the user. A hose extends between the gas flow generator and the mask for providing a flow of gas. The system has a mechanism for turning the flow of gas on at a time distinct from turning on the apparatus.

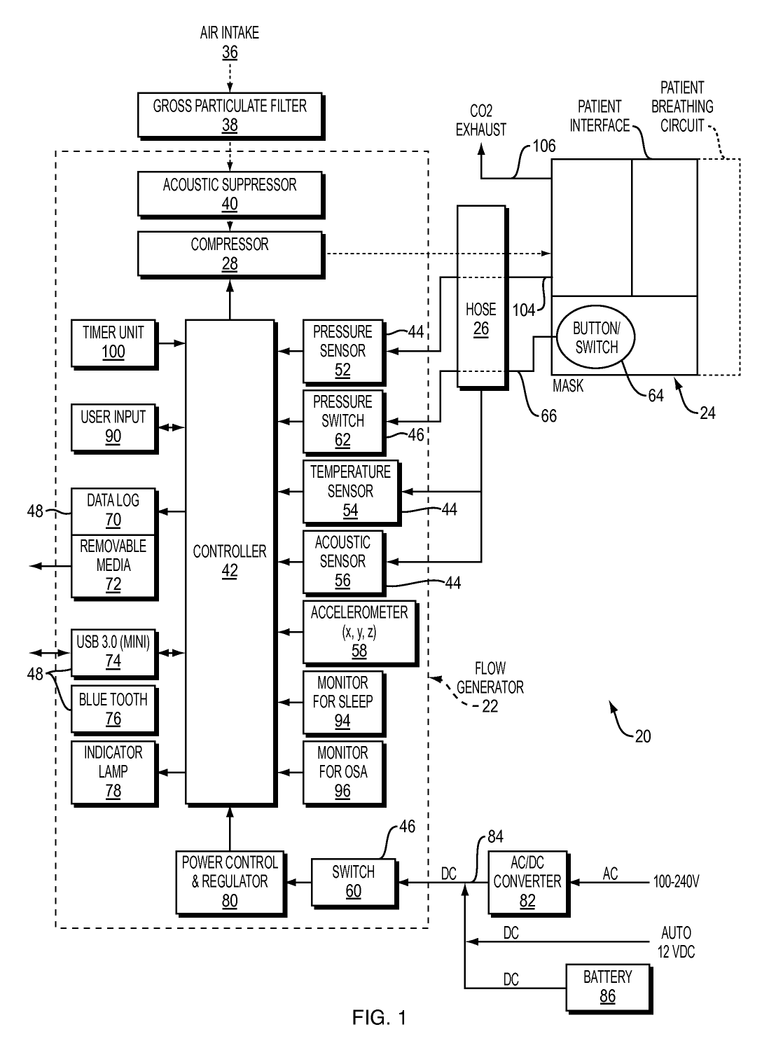

Referring to FIG. 1, a schematic of a CPAP system 20 with a sleep activity control system is shown. The system 20 has a gas flow generator 22, also referred to as a blower unit, for providing a source of pressurized breathable air, a patient interface 24, such as a mask, that is removably worn by the patient, and an interconnector 26, such as a hose. The flow generator 22 has a compressor 28 for taking ambient air and creating pressurized air flow. The pressure range desired can vary, but generally falls in the range of between 4 and 20 centimeters of water. The range of the system 20 can extend even higher from 0 centimeters of water to 30 or 50 centimeters of water. The average user/patient typically is treated by a pressure of between 6 and 14 centimeter of water. A typically user utilizes an air flow rate of 20 to 60 liters of air per minute.

The air for the mask 24 is drawn in at an air intake 36 and passes through a filter 38 and an acoustic suppressor 40 prior to the blades of the impeller of the compressor 28. The compressor 28 compresses the air, thereby increasing the pressure; an expansion chamber of the compressor allows the compressed air to expand and increase the velocity of the air. The pressurized air passes through the interconnector 26 to the mask 24.

The flow generator 22 in addition has a controller 42 and a plurality of sensors 44, switches 46, and interface devices 48 for controlling the compressor 28.

The sensors 44 can include a pressure sensor 52 that monitors the pressure of the air in the flow generator 22, the interconnector 26, and/or the mask 24. The sensors 44 can also include a temperature sensor 54, an acoustic sensor 56, and an accelerometer 58. The plurality of switches 46 includes a switch 60 for the system 20 located on the flow generator 22. In addition, the system 20 has a pressure switch 62 which connects to a switch 64 on the mask 24 with a conduit 66 carried by the interconnector 26.

The interface devices 48 include a data log 70 associated with removable media 72. The interface devices 48 can also include a USB port 74, blue tooth 76, and an indicator lamp 78.

Still referring to FIG. 1, the flow generator 22 has a power control and regulator 80 interposed between the switch 60 and the controller 42. The system 20 can be powered by various methods as represented by the AC/DC converter 82, a DC output 84 such as from an auto, and/or a battery 86. While 12 volts DC is shown, it recognized that the system may receive power inputs at a different voltages such as 14-15 volts, 19 volts, or 24 volts.

The system 20 has a user input 90 that allows the user/clinician to select/modify the working of the system 20. For example, the clinician can adjust the pressures or mode of treatment. The mode could include mono-level CPAP, bi-level CPAP, and ramping. The user can select for example when the blower turns on as described in the paragraph below.

In addition, the flow generator 22 has a timer unit 100 that is capable of controlling when the compressor 28 is on and providing pressured air to the patient interface, mask 24 through the interconnector 26. In addition, the flow generator 22 in certain embodiments has an interface device 94 for detecting and monitoring sleep stages; as explained in more detail below, the interface device takes input from a sensor and determines if the user is asleep. In addition in certain embodiments, the flow generator 22 has a second or alternative interface device 96 for monitoring for detecting obstructed sleep apnea. The timer unit 100, the interface device 94 for detecting sleep stage, and the interface device 96 for detecting OSA is described in provisional application 61/559,912 filed on Nov. 15, 2011 which is incorporated herein by reference.

The mask 22 is most commonly a nasal mask or a full face mask as shown. It is recognized that the patient interface 22 can be other devices such as a nasal cannulae, an endotracheal tube, or any other interface, as explained below, based on other suitable appliances for interfacing between a source of breathing gas and a patient.

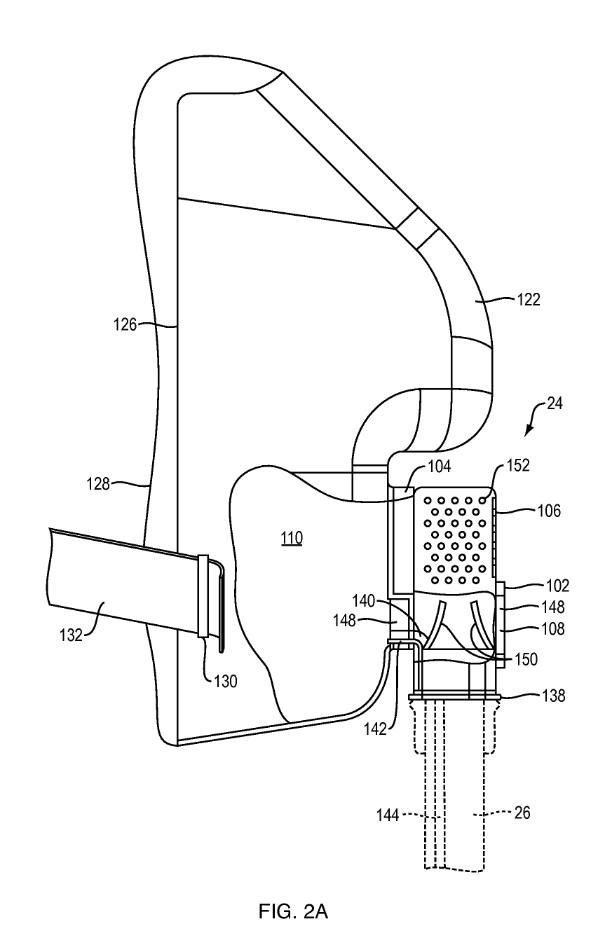

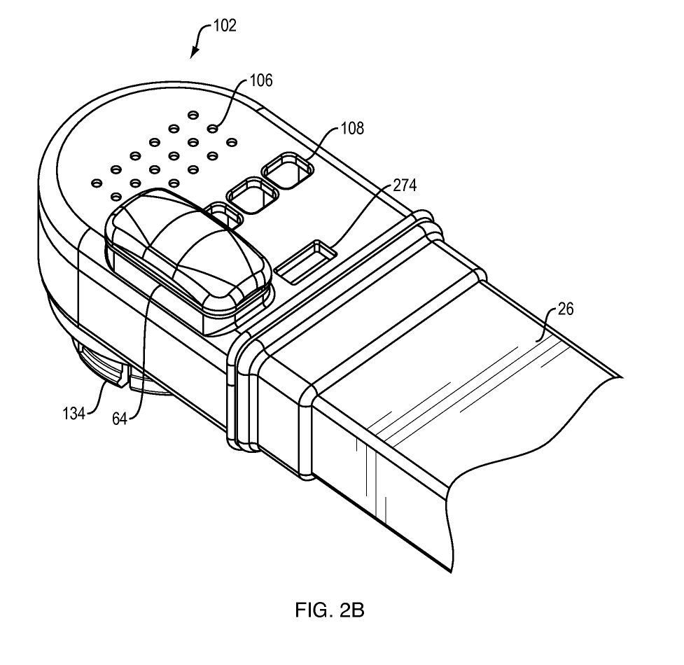

Referring to FIG. 2A, a sectional view of a portion of the mask 24 is shown. The mask 24 has a shell or frame 122 with a gas inlet aperture 104. The shell 122 has a rim portion 126. The mask 22 has a pliable portion 128 such as a cushion or gel portion that is received on the rim portion 126 of the shell 122. The frame 122 has a plurality of connection points 130 for connecting a plurality of straps 132 to retain the mask 24 to the user's face.

The mask 24 has a mask connector 102 with a connector 134, as best seen in FIG. 2B, that interfaces with the gas inlet aperture 104. The mask connector 102 has an interface 138 for receiving the hose 26, shown in hidden line in FIG. 2A, for receiving pressurized gas from the flow generator 22. In addition, the mask 24 has a suitable exhaust port 106, schematically indicated in FIG. 1, and shown in FIG. 2A in the mask connector 102 for exhausting of breathing gases during expiration. The exhaust port 106 is some time referred to as a washout vent.

Exhaust port 106 preferably is a continuously open port which imposes a suitable flow resistance upon exhaust gas flow to permit a pressure controller system 140 including a port 142 in the mask, a conduit 144, shown in hidden line, through the hose 26 to the pressure sensor 52 which through the controller 42, as seen in FIG. 1, controls the pressure of air flow from the compressor 28 to the mask 24.

In one embodiment, the exhaust port 106 may be of sufficient cross-sectional flow area to sustain a continuous exhaust flow of approximately 15 liters per minute. The flow via the exhaust port 106 is one component, and typically the major component of the overall system leakage, which is an important parameter of system operation.

In addition still referring to FIG. 2A, the mask 22 has a breathing port 108 that is open when the compressor 28 is not providing pressurized air at a specific rate. The breathing port 108 includes a plurality of the opening 148 and a pair of flaps 150 that cover the opening 148 when pressurized area is flowing from the hose 26 to the mask connector 102.

Still referring to FIG. 2A, the system 20 has a heat moisture exchange (HME) component 210. The HME component 210 carried by the mask connector 102 collects moisture as the user exhales that passes through the exhaust port 106. As the user receives pressured air from the flow generator 22 through the mask connector 102, the pressurized air flows through the HME 210 to the cavity 110 with the shell 202 of the mask 24 therein providing moisture to the air.

Referring to FIG. 2B, the mask connector 102 with the hose 26 is shown. The mask connector 102 has the exhaust port 106 and the breathing port 108. In addition, the mask connector 102 has the button, the switch 64 that is connected to the pressure switch 62 for turning on the compressor 28.

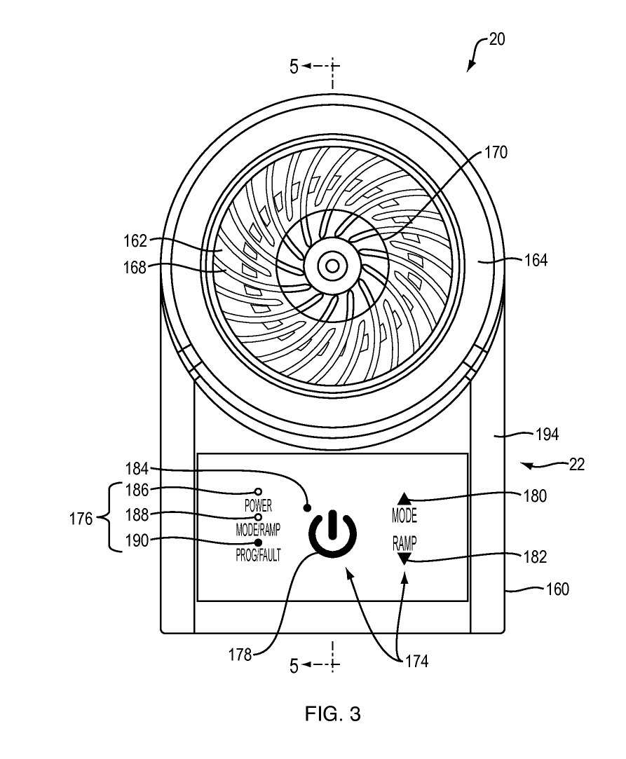

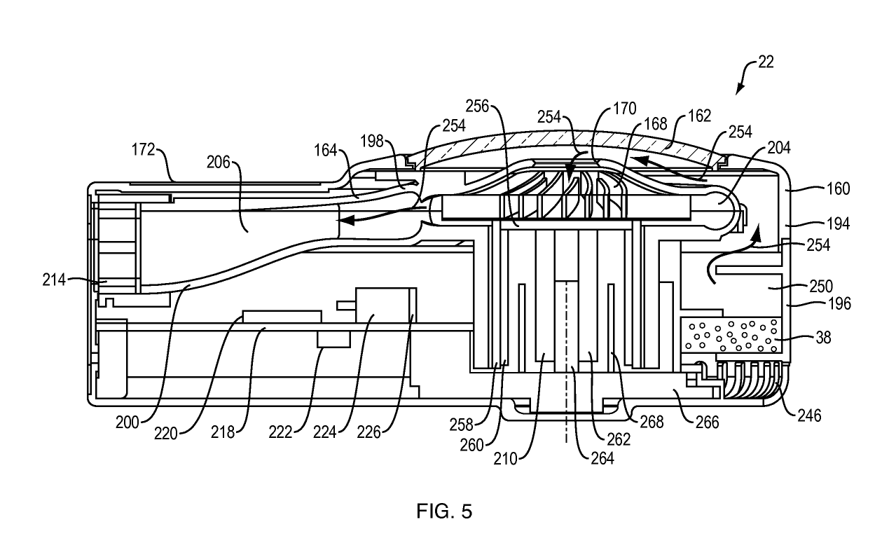

Referring to FIG. 3, a top view of the blower unit/flow generator unit 22 is shown. The flow generator 22 as indicated above takes air and compresses the air to create a pressurized gas (air) that can be delivered to the patient interface 24, such as a mask at a pressure between 4 and 20 centimeters of water and at a flow rate of between 20 to 60 liters of air per minute in an embodiment. The flow generator 22 has a housing 160 with a translucent dome 162. In addition, the flow generator 22 has a casing 164, which in the embodiment shown is transparent, showing an impeller 168 of the compressor 28. The casing 164 has an opening 170 through which air flows as explained in greater detail with respect to FIG. 5. The flow generator 22 has a first input/output portion 172 which has a plurality of switches 174 and a plurality of indicators 176. In the embodiment shown, the first input/output portion 172 is a membrane switch 172 having three switches 178, 180, and 182 and four LED indicators 184, 186, 188, and 190.

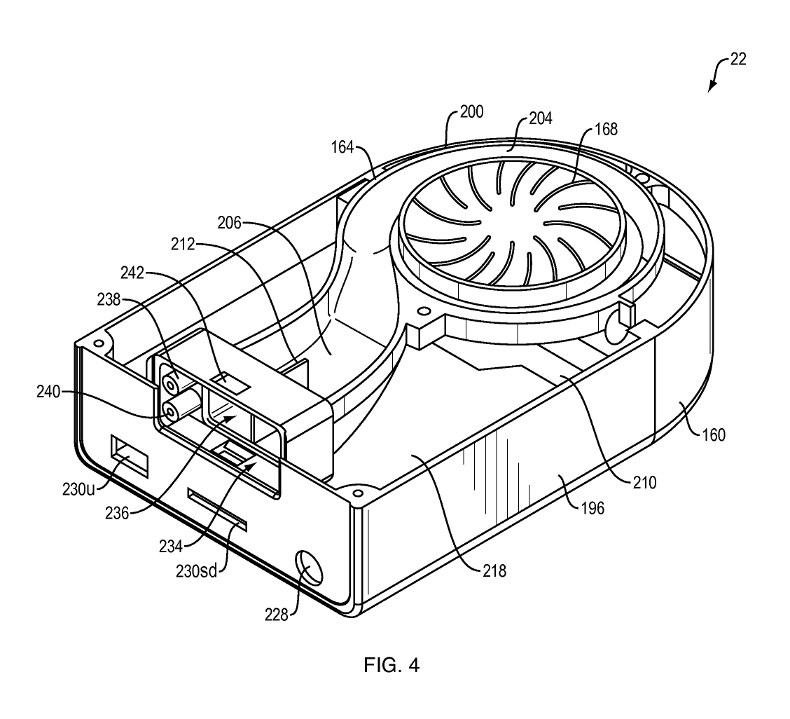

Referring to FIG. 4, an isometric view of the flow generator 22 with a portion removed is shown. The housing 160 has an upper shell 194, seen in FIG. 3, which is removed in FIG. 4 and a lower shell 196. The casing 164 has an upper portion 198, which is removed in FIG. 4, and a lower portion 200. The casing 164 defines a collection chamber 204 of the compressor 28 which encircles the impeller 168. As the impeller 168 rotates counter-clockwise, the air is pushed into the collection chamber 204 and moves into an expansion chamber 206 as defined by the casing 164. Underlying the impeller 168 is a motor 210 of the compressor 28.

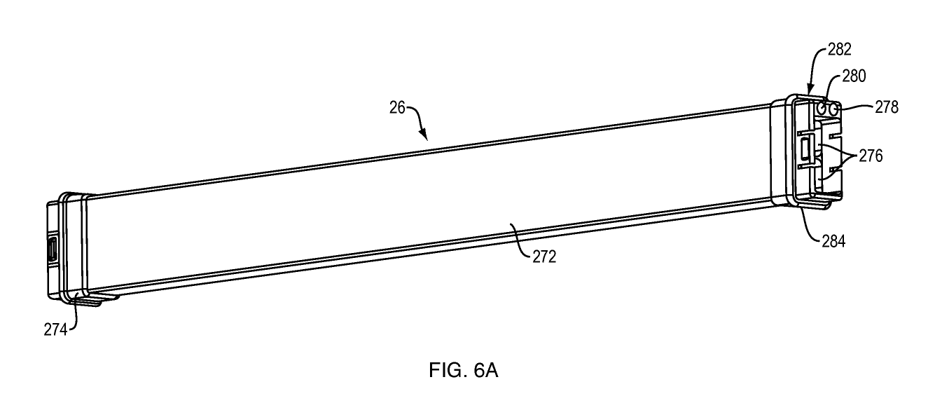

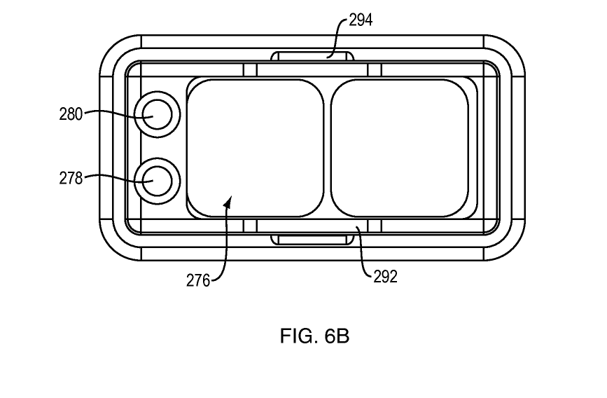

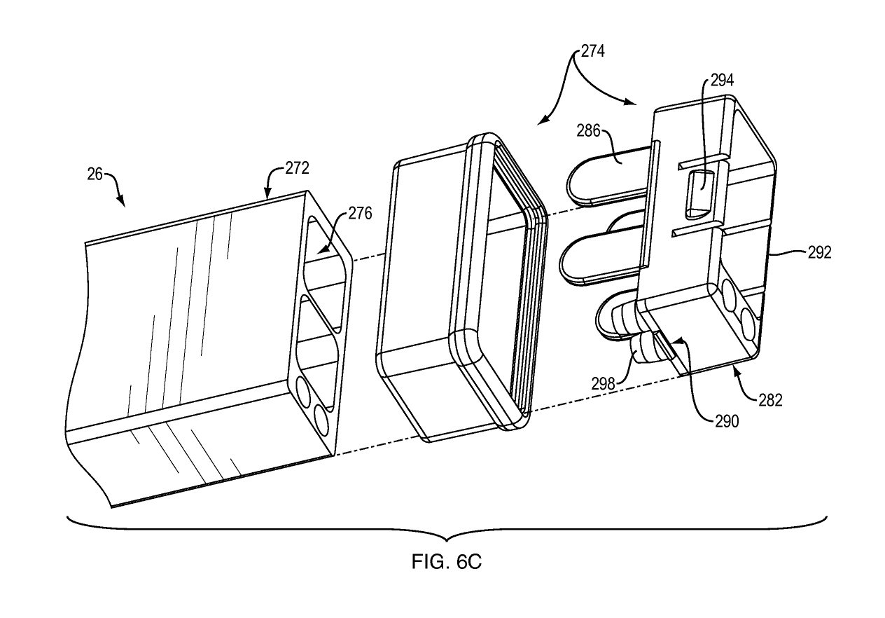

Still referring to FIG. 4, the casing 164 at the expansion chamber 206 end has a fin 212 that splits the flow of air into two portions. The housing 160 has a hose interface connector 214 that interfaces with the hose 26 described in more detail below with respect to FIGS. 6A, 6B, and 6C. The flow generator 22 has a printed circuit board (PCB) 218 that contains the circuitry to both monitor the inputs and control the motor 210. The PCB 218 has a variety of components including a motor control integrated circuit, an orientation sensor, a pressure switch, and a pressure sensor. In addition, mounted on the PCB 218 are a power connector 228 and a pair of data connectors 230 in the embodiment shown. The first data connector is a mini USB receptacle 230u and the second data connector is a micro sd card reader 230s.

The hose interface connector 214 of the housing 160 has a generally rectangular opening that receives the hose 26. The hose interface connector 214 has an opening 234 that opens up onto an air flow hole 236 that receives the end of the casing 164. In addition the connector 214 has a pair of projections 238 that are received by the hose 26. Each projection 238 has an opening 240 that is in communication with a sensor or switch. In addition, the hose interface connector 214 has a pair of detent openings 242 for securing the hose 26.

Referring to FIG. 5, a sectional view of the flow generator 22 taken along line 5-5 of FIG. 3 is shown. The housing 160 with the translucent dome 162 of the flow generator 22 encases the casing 164 that defines the collection chamber 204 and the expansion chamber 206. The end of the casing 170 is shown fitted into the hose interconnection connector 214. The PCB 218 has various components 244 including a motor control integrated circuit 220, an orientation sensor 222, a pressure switch 224, and a pressure sensor 226 as seen in the Appendix.

The flow generator 22 has a series of slots 246 in the shell 160 defining an intake 248 through which it draws in ambient air. The air is drawn through a series of baffle chambers 250 defined by the shell 246 and used to suppress noise. Located in the baffle chamber 250 is a filter 38 for blocking particulate that may be in the air. The air flows out the baffle chamber 250 and between the casing 164 and the upper shell 194 including the translucent dome 162 and is drawn through the opening 170 in the casing 164. The impeller 168, which is enclosed in the casing 170, as it rotates forces the air into the collection chamber 204. The collection chamber 204 increases in size as it encircles the impeller 168 in the counterclockwise direction as seen in FIGS. 4 and 5. The pressurized air expands in the expansion chamber 206 as it moves to the hose interface connector 204. Arrows 254 show the flow of the air through the flow generator 22.

The motor 210 that drives the impeller 168 has an upper portion 256 with an outer sleeve 258 that encircles a magnet 260. The upper portion 256 is held in position by an air bearing sleeve 262 encircling a pin 264 projecting upward from a motor board 266. The motor board also has a coreless waveform continuation coil 268 that receives current in a manner that creates a field to influence the magnet and rotates the upper portion 256 of the motor and the impeller 168.

In an embodiment, the flow generator 22 is approximately 4 inches by 2½ inches by 1½ inches in size. The weight of the flow generator 22 is less than 8 ounces.

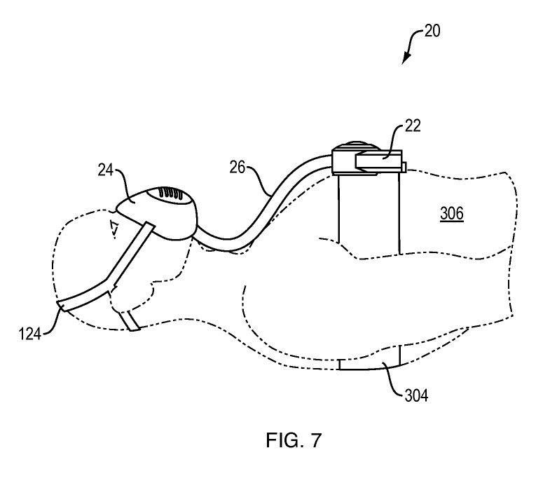

Referring to FIG. 6A, an isometric view of the hose 26 is shown. The hose 26 has a tube (hosepipe) 272 and a hose connector 274 at each end. The hose 26 has a pair of air flow channels 276 for communicating the pressurized air from the flow generator 22 to the mask 24 as seen in FIGS. 1 and 7. In addition, the hose 26 has a pair of communication channels 278 and 280. In the embodiment shown, one of the communication channels 278 connects the button 64, as seen in FIG. 2B, on the mask 24 and the pressure switch 62, as seen in FIG. 1, to allow the user to turn the system 20 from a stand-by mode to operation. The other communication channel 280 connects a port 142, as seen in FIG. 2A, on the mask 24 with the pressure sensor 52 located on the PCB 218 in the flow generator 22; the pressure sensor 52 monitors the flow and allows the controller 42 to adjust parameters as discussed below.

FIG. 6B is a side view of the hose 26 showing one of the hose connectors 274. FIG. 6C shows the hose 26 exploded with the two components of the hose connector 274 separated. The hose connector 274 has a mating portion 282 and an outer sleeve 284. The mating portion 282 has a plurality of tabs 286 that are received in the flow channels 276 of the hosepipe 272. The outer sleeve 284 encircles the edge of the hosepipe 272 and has a groove to receive a ridge 292 located on the mating portion 282 to assist in securing the components. The mating portion 282 has a pair of tabs 292. Each tab 292 has a detent 294 that is received in one of the detent openings 242 located on the hose interface connector 214 of the flow generator 22 as seen in FIG. 4 or on the mask connector 102 as seen in FIG. 2B. The projections 238 as seen in FIG. 4 are received in communication channels 278 and 280. The mating portion 282 has projections 298 which are received in the respective openings in the hosepipe 272

When the user is ready to use the CPAP system 20, he turns on the system 20 by turning on the switch as represented by block 60 in FIG. 1. This action places the unit into a stand-by mode. The system 20 can operate in several different modes. While some of the operations are described separately, it is recognized that one or more of the modes of operation can be used concurrently.

The abbreviation CPAP stands for continuous positive air pressure which in generic terms is a method of noninvasive or invasive ventilation assisted by a flow of air delivered at a positive pressure throughout the respiratory cycle. It is performed for patients who can initiate their own respirations but who are not able to maintain adequate arterial oxygen levels without assistance. Sometimes the word “continuous” is replaced with the “constant.” For the purpose of this patent, constant positive airway pressure is referred to as mono-level CPAP. CPAP can be in various modes including mono-level CPAP, Bi-level CPAP, Auto-PAP, Servo-ventilation, and ramping.

In a mode of operation, the user places the mask 24 on his face. In one mode, the user presses the button 64 on the mask 24 and the system 20 goes immediately into operation. The mode of operation once the switch is pressed includes an open-loop mode or a closed-loop mode. The modes of operation are described in greater detail below.

In another mode, the compressor 28 is not turned on until a later time. The later time can be based on a timer, detection of sleep, or detection of OSA. The time delay, detection of sleep, or detection of OSA to turn on the compressor 28 is described in 61,559,912 filed on Nov. 15, 2011 which is incorporated herein by reference.

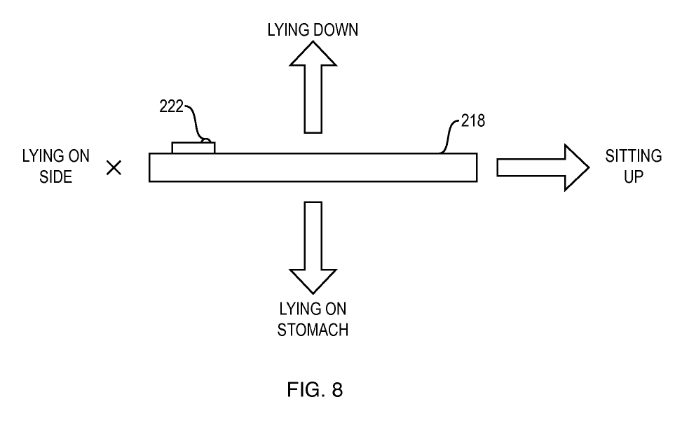

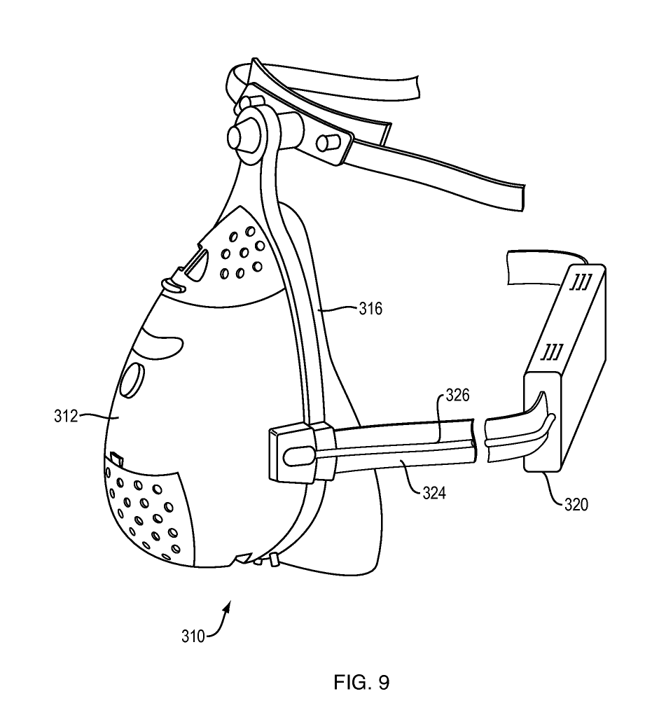

In another mode or in combination with one or more modes above, the system has an orientation sensor 222 such as a tilt sensor or an accelerometer to determine the orientation of the system 20. The orientation sensor 222 can be located on the mask 24 or in the flow generator 22. As described below with respect to FIGS. 7, 8, and 11-16, the location of the of flow generator 22 can be adjacent to the user's chest, laying next to the user such as on the bed mattress, or on a night stand or table adjacent to the bed. When the flow generator 22 is attached to the body of the user, such as affixed to the user's chest 306 by one or more straps 304 as shown in FIG. 7 or mounted to the mask 24 or straps 122 for the mask 24 as shown in FIG. 9, the orientation sensor 222 can be located in the flow generator 22 such as represented by the printed circuit board 218 in FIG. 8. In other situations such where the flow generator 22 is located on a night stand, the placement of the orientation sensor 222 in the mask 24 achieves a better result.

The orientation sensor 222 provides input to the controller 42 when the unit 22 is oriented in a vertical direction, such as when a user has sat up or stood up as represented by the arrow pointing to the right in FIG. 8. The system 20 can shut off the compressor 28 when the user is in this position. As indicated above, the user may choose not to select this mode for example if they are using the system while flying on a commercial airline or other type of vehicle such as a train; the sensor 222 can be adjusted to operate at different angles to compensate for inclined beds or other situations.

In addition, the orientation sensor 222 in addition can determine if the user is lying on their back, stomach, or lying on their side as represented by various arrows in FIG. 8. When the user is lying on their side the arrows are into or out of the page. In that the person's orientation effects the obstruction that causes sleep apnea, the amount of pressure needed varies.

In OSA, the upper airway collapses and blocks airflow during sleep. While the collapse can occur at several points, for example the soft palate in the upper oropharyngeal or pharynx level is drawn downward into the throat during sleep and blocks the airway, the orientation of the user and gravity effects can influence the percentage of blockage.

Referring back to FIGS. 4 and 5, the circuitry on the printed circuit board (PCB) 218 provides controlled output to the motor 218 that is used to rotate the impeller 168. In the embodiment shown in FIGS. 4 and 5, there are the pressure sensor 52, the pressure switch 224, which can be a pressure sensor, and the orientation sensor 222, which are used in the control of the compressor 28 in addition to the membrane switch 172. As indicated above, the membrane switch 172 shown in FIG. 3 has three input (membrane momentary) switches 178, 180, and 182 and four indicators in the forms of LEDs 184, 186, 188, and 190.

The inputs allow the system 20 to operate in various modes including a closed loop feedback between the pressure sensor 52 located on the PCB 218 and the motor control integrated circuit 220 to permit regulated pressure output of the compressor 28 using the motor 210 RPMs and the reading of the pressure output in the mask 24 as described above with respect to FIG. 2A.

As indicated above, a pressure switch 224, which is in the embodiment a pressure sensor, reads a pressure signal from a small remote pneumatic pump, the button 64 shown in FIG. 2B. The pneumatic pump is simply a small rubber bulb that when squeezed, provides an elevated pressure signal to the switch. This acts as an input into the circuitry on the PCB 218.

The motor 210 drives the impeller 168 of the compressor 28 which provides pressurized air to deliver to the user's (patient) respiratory circuit. The operation requires that the circuitry on the printed circuit board (PCB) 218 functions in an open-loop or closed-loop mode. The closed-loop mode regulates the pressure delivered by the compressor 28 to the user. The user and his lung, nose, mouth, pharynx and other body elements are sometimes known as the patient circuit. Open-loop only controls the motor 210 at a set RPM and ignores inputs from the pressure sensor 52.

In operation, the compressor 28 is activated by user controls and, in certain modes, inputs from the orientation sensor 222. These controls/inputs are momentary switches 174, the pressure switch 224, and the orientation sensor 222. In certain embodiments, such as the embodiment shown, the connection of the flow generator 22 to power provides power to the printed circuit board 218 and places the flow generator 22 into stand-by mode. The momentary switch 174 inputs are momentary closure and select a mode for the compressor 28 to power up which produces pressure. Table 3 shows an example of the operation of the membrane. The pressure switch 224 reads an elevated pressure signal from a small remote pneumatic pump. The pressure switch 224 input and the momentary switch 174 input are the same and can be used on an either/or basis. The input from the orientation sensor 222 simply pauses or resumes operation of pressure when it is tilted in certain embodiments.

Table 1 shows various forms of control of the system. As indicated above, the plugging of the blower unit 24 into a power source places the unit 24 into a standby mode.

TABLE 1

| Operation | Input Type | Resulting Action |
|---|---|---|
| Power | Power | PCB is powered and in standby mode |
| applied | connector | |
| | (228) | |
| Select mode | Switch (174) | Selects one of two modes for pressure |
| | | feed-back and also selects on/off state for |
| | | Auto-Tilt Operation. The mode switch |
| | | toggles from one mode to another. |
| Start | Switch (174) | Input to MC (micro-controller) to run |
| treatment | | current motor instructions required by the |
| | | mode that is selected |
| Start | Pressure | Input to MC from pressure switch is |
| treatment | Switch (224) | identical to input command from electrical |
| | | switch. The two signals are either/or. |
| Pause | Orientation | Outputs signal to MC which determines if |
| Treatment | Sensor (222) | signal is in a range to pause or resume |
| | | motor/compressor instructions. |
| Sleep | Logic | Turns on flow when sleep or respiratory |
| activation | | burden is detected. |

As indicated above, the system can be operated in several modes including an open loop mode and a closed loop mode. In the open loop control mode, the operation of the motor 210 is set to a specified RPM (revolutions per minute). The RPM is dictated by a look-up table of RPMs. In this embodiment, the system 20 does not take feedback from the pressure sensor 52 in this mode of operation.

In the closed loop mode, the system 20 uses the pressure sensor 52 to regulate the pressure output of the compressor 28. The circuitry adjusts the speed, the RPM, of the motor 210 by comparing the pressure sensed by the pressure sensor 52 as described above with respect to FIGS. 1 and 2. In a preferred embodiment, the system adjusts the RPM of the motor 210 so that the pressure within the mask 24 is within 0.5 cm H₂O of the set value.

As indicated above, the system 20 has an orientation sensor 222. The orientation sensor 222 serves two functions. The first function is to pause or resume the compressor 28 including the motor 210 and the impeller 168. The printed circuit board 218 is located in the flow generator 22 that in certain embodiments is strapped to a patient's chest such as shown in FIG. 7. While the user is in a prone position (laying down) the system 20 operates normally. When the user rises (sits up—upright position) the compressor 28 pauses. Programming the unit by the user is permitted at any time, but if upright, the compressor 28 will not operate. The user can turn off this function. For example, a user on an airplane may desire that the system run even when the user is sitting up.

As indicated above, the orientation sensor 222 in addition can determine when the user is asleep such as lying on his or her back or lying on their side as discussed above with respect to FIG. 8. In contrast to above where the orientation sensor 222 turns the system on and off, the system adjusts the motor speed by varying the RPM therein adjusting the pressure.

As indicated above, the system can operate in various modes. The following are examples of various modes. In one of the open loop modes of operation, the system 20 can be placed in a discrete pressure mode that allows the clinician to select from programmed pressures from 4-30 cm H₂O pressure. The mode is only operated in the open-loop control mode which instructs the motor 210 to operate at a specific RPM.

In one of the closed loop modes of operation, the system can be placed in another discrete mode. In this discrete mode, the user or the clinician can select from one of 5 pre-set pressure settings. In contrast to the open loop mode addressed above where the RPMs are set, in this mode the system uses feedback from the pressure sensor 52 to maintain the pressure level selected by the user input. The user pressure setting input is performed through selection of a pressure pre-set.

An example of the pre-set pressure references to instruct the closed-loop control to output this same pressure using the pressure sensor as feedback is shown on Table 2.

TABLE 2
Closed Loop Pre-Set Pressure Table

| Pre-set | PRESSURE | | |
|---|---|---|---|
| label | (absolute) | REFERENCE | BEHAVIOR |
| 1 | 4 | Uses pressure feed- | Motor RPMs are changed |
| | | back to regulate | dynamically to increase or |
| | | desired pressure. | decrease pressure output to |
| | | | active desired setting |
| 2 | 10 | Uses pressure feed- | Motor RPMs are changed |
| | | back to regulate | dynamically to increase or |
| | | desired pressure. | decrease pressure output to |
| | | | active desired setting |
| 3 | 15 | Uses pressure feed- | Motor RPMs are changed |
| | | back to regulate | dynamically to increase or |
| | | desired pressure. | decrease pressure output to |
| | | | active desired setting |
| 4 | 20 | Uses pressure feed- | Motor RPMs are changed |
| | | back to regulate | dynamically to increase or |
| | | desired pressure. | decrease pressure output to |
| | | | active desired setting |
| 5 | 30 | Uses pressure feed- | Motor RPMs are changed |
| | | back to regulate | dynamically to increase or |
| | | desired pressure. | decrease pressure output to |
| | | | active desired setting |

As indicated above, in certain embodiments the user can use a first input/output (membrane) 172 as seen in FIG. 3 to both input controls and monitor the status of the system 20. The below table, Table 3, shows an example of input and output. While the relation of switches 174 and indicators (LED) 176 can be adjusted, the following is one relationship: B1—is On/off power switch 178; B2—is mode switch 180; and B3—is ramp switch 182. L1 is LED 184; L2 is LED 186; L3 is LED 188; and L4 is LED 190.

| | CLOSURE | | | |
|---|---|---|---|---|
| BUTTON | TIME | MODE | LAMP | BEHAVIOR |
| NOTES | — | — | — | All functions activate after momentary buttons are released |
| — | — | Power present | L2 | Lamp “on” constant |
| — | — | Stand-by | L1 | Lamp blinks 0.5 sec intervals |
| — | — | Program/data/fault | L4 | Lamp blinks 0.5 sec intervals for data during transmit/receive. |
| | | | | Also lamp blinks 0.5 sec intervals during a fault condition until |
| | | | | reset. |
| B1 | 0.5-1.5 sec | Pressure “on” | L1 | Lamp “on” constant. Switch toggles between “pressure on and |
| | | | | off” |
| B1 | 0.5-1.5 sec | Pressure “off” | L1 | Lamp “off”. Switch toggles between “pressure on and off” |
| B1 | 2-3 sec | Auto | L2 | Lamp blinks 0.5 sec intervals |
| B2 | 0.5-1.5 sec | Open-loop | L3 | Lamp “on” constant. Switch toggles between “modes” |
| B2 | 0.5-1.5 sec | Closed-loop | L3 | Lamp “off”. Switch toggles between “modes” |
| B2 + B3 | 0.5-1.5 sec | Change pre-set | L3 + | Lamps blink at 0.5 sec intervals. This is pre-set pressure set |
| | | pressure | L4 | mode. The mode is “on”. Switches are briefly closed |
| | | | | simultaneously. The simultaneous closure is used to activate |
| | | | | “on” and then “off”. |
| B2 + B3 | 0.5-1.5 sec | Change pre-set | L3 + | Pre-set pressure set mode is “off”. Lamps are “off”. Switches |
| | | pressure | L4 | are closed simultaneously to exit the pre-set pressure set mode. |
| | | | | The simultaneous closure is used to flash/toggle “on” and then |
| | | | | “off”. |
| B2 | 0.5-1.5 sec | Change pre-set | L3 + | Pre-set pressure set mode is “on”. Lamps blink at 0.5 sec |
| | | pressure | L4 | intervals. After entering pre-set pressure set mode, B2 is used |
| | | | | to increment pressure pre-sets by increments of 1. There are 5 |
| | | | | pre-sets: 4, 10, 15, 20, and 30 cmH20 pressures (see table |
| | | | | above). |
| B3 | 0.5-1.5 sec | Change pre-set | L3 + | Pre-set pressure set mode is “on”. Lamps blink at 0.5 sec |
| | | pressure | L4 | intervals. After entering pre-set pressure set mode (above), B3 |
| | | | | is used to decrement pressure pre-sets by increments of 1. |
| — | — | Pre-set label | L4 | Lamp blinks at 0.5 sec intervals after pre-set pressure set mode |
| | | count | | is exited. The lamp blinks 1 illumination for each count to |
| | | | | represent the pre-set. Example: 3 blinks would indicate that the |
| | | | | pre-set is “3” or 15 cmH2O setting. |
| B1 + B2 | 0.5-1.5 sec | Discrete | L3 + | Lamps blink at 0.2 sec intervals. This is discrete pressure set |
| | | pressure set | L4 | mode (different from pre-set pressure mode above). Switches |
| | | | | are closed simultaneously. The simultaneous closure is used to |
| | | | | activate “on” and then “off”. |
| B1 + B2 | 0.5-1.5 sec | Discrete | L3 + | Lamps “off”. Discrete pressure set mode is “off”. Switches are |
| | | pressure set | L4 | closed simultaneously to exit the pressure set mode. The |
| | | | | simultaneous closure is used to “on” and then “off”. |
| B2 | 0.5-1.5 sec | Discrete | L3 + | Discrete pressure set mode is “on”. Lamps blink at 0.2 sec |
| | | pressure set | L4 | intervals. After entering pressure set mode (above), B2 is used |
| | | | | to increment pressure pre-sets by increments of 1. There are 4 |
| | | | | pre-sets: 4, 10, 15, 20 cmH20 pressure. |
| B3 | 0.5-1.5 sec | Discrete | L3 + | Discrete pressure set mode is “on”. Lamps blink at 0.2 sec |
| | | pressure set | L4 | intervals. After entering pressure set mode (above), B3 is used |
| | | | | to decrement pressure pre-sets by increments of 1. There are 4 |
| | | | | pre-sets: 4, 10, 15, 20 cmH20 pressure. |
| — | — | Discreet | L4 | Lamp blinks at 0.5 sec intervals after discrete pressure set |
| | | pressure setting | | mode is exited. The lamp blinks 1 illumination for each count |
| | | count | | to represent the pressure setting. Example: 8 blinks would |
| | | | | indicate that the pressure is set to 8 cmH2O. |

Table 3 shows an example of the operation of teh membrane

As indicated above, the system 20 can have various interface devices 48 as shown in FIG. 1 including a USB connector 74 and removable media 72 such as mini sd card 230sd shown in FIG. 4. These interface devices 48 can work in conjunction with the first input/output portion (membrane) 172 or as an alternative.

Referring to FIG. 9, an integrated CPAP system 310 is shown. The integrated CPAP system 310 includes a flow generator, a blower 312 which is either connected to or encased in a rigid mask shell 316 and covered with a flow generator cap.

A power supply enclosure 320, which may include batteries, is connected via a strap 324 to the integrated CPAP unit 310. The strap 324 may be adjustable such that the power supply 320 may be supported at the back of the user's neck. While a preferred location is on the back of the neck, other locations, such as the arm, shoulder, hip, or chest etc. may be used. In one embodiment, a cooling supply conduit 326 supplies gas from the integrated CPAP unit 310 to the power supply 320.

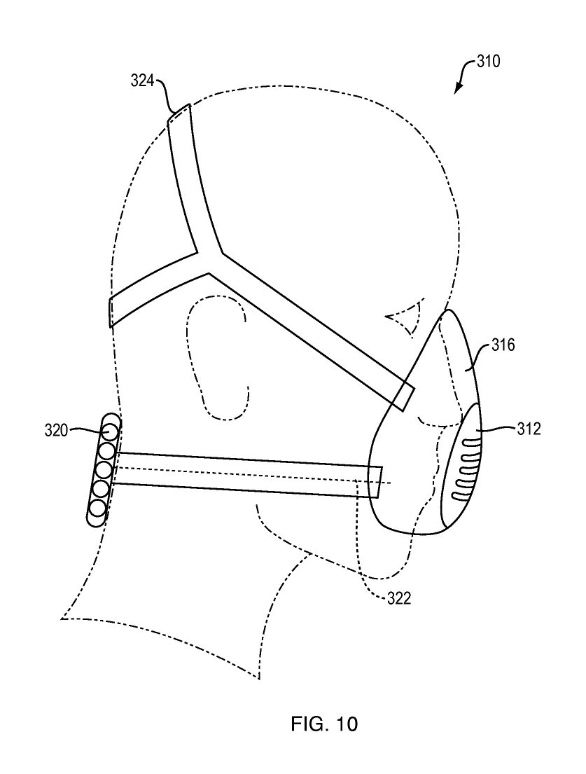

Referring to FIG. 10, a side view of a user wearing an alternative integrated system 310 is shown. The power supply, a plurality of batteries, is shown on the back of the user's neck.

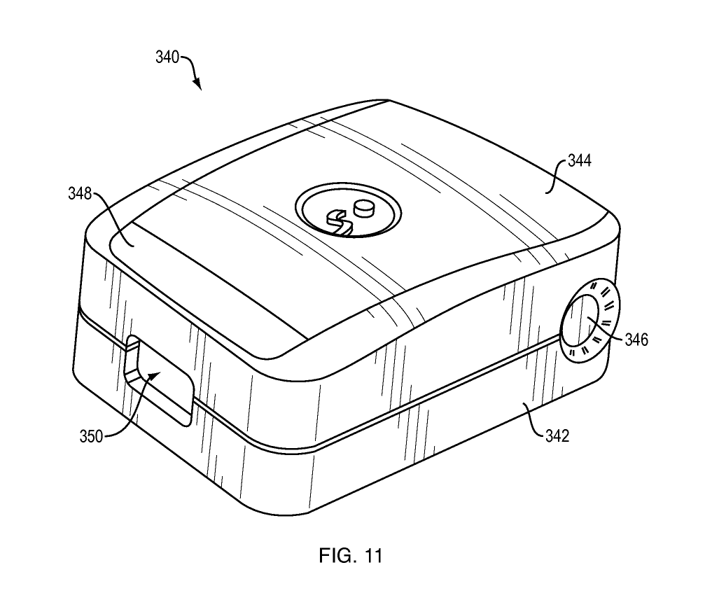

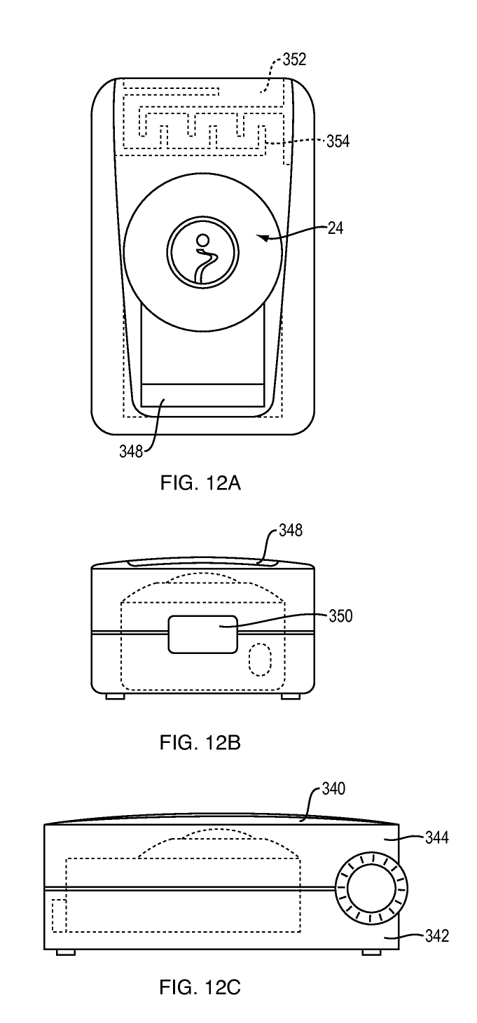

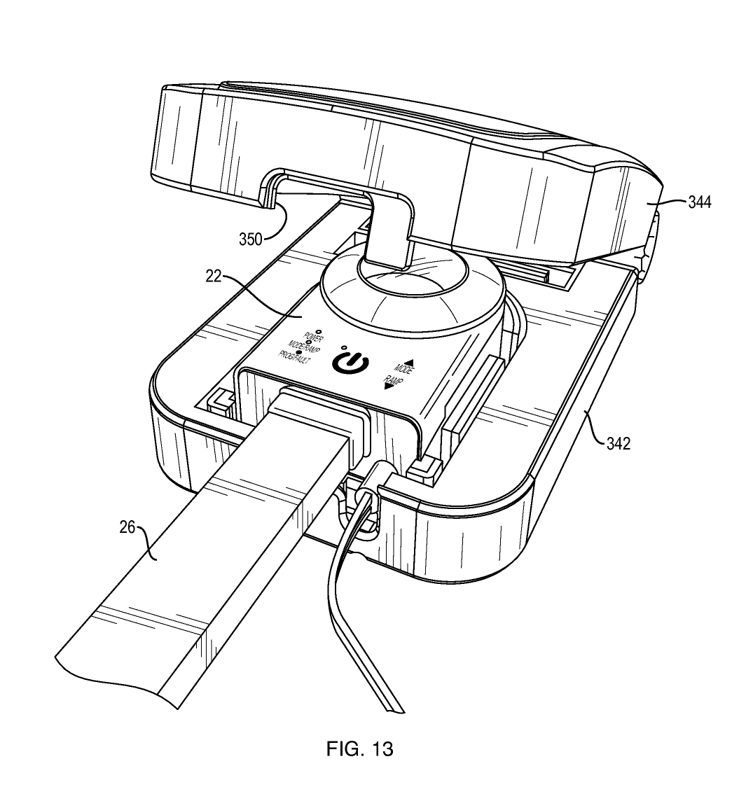

As indicated above, the blower unit or flow generator 22 can be located at various locations including strapped to the chest 306 as seen in FIG. 7. In addition, the flow generator 22 can be placed in a docking station 340 as shown in FIG. 11 and FIGS. 12A-12C. The docking station 340 shown in perspective in FIG. 11 has a lower clam shell 342 and an upper clam shell 344 that enclose the flow generator 22. The docking station 340 has a hinge, a pivot point 346 that allows the upper clam shell 344 to pivot upward relative to the lower clam shell 342. The upper clam shell 344 in one embodiment has a single large button 348 for turning the compressor 28 on and off. The button 348 acts similarly to the button 64 on the mask 24, as seen in FIG. 2B. The clam shells 342 and 344 of the docking station 340 form an opening 350 through which the hose 26 can connect to the flow generator 22 as shown in FIG. 13.

Referring to FIGS. 12A-12C, a top view, a front view, and a side view of the docking station are shown. The top view, FIG. 12A, shows the blower unit 24 in phantom line in the docking station 340. In addition, a plurality of chambers 352 formed by baffling 354 in the docking station 340 are shown in hidden line. The baffling 354 is used to reduce the noise of the air being drawn into the compressor.

Referring to FIG. 13, a perspective view of the docking station 340 with the upper clam shell 344 hinged upward is shown. The hose 26 is shown passing through the opening 350 in the clam shells 342 and 344.

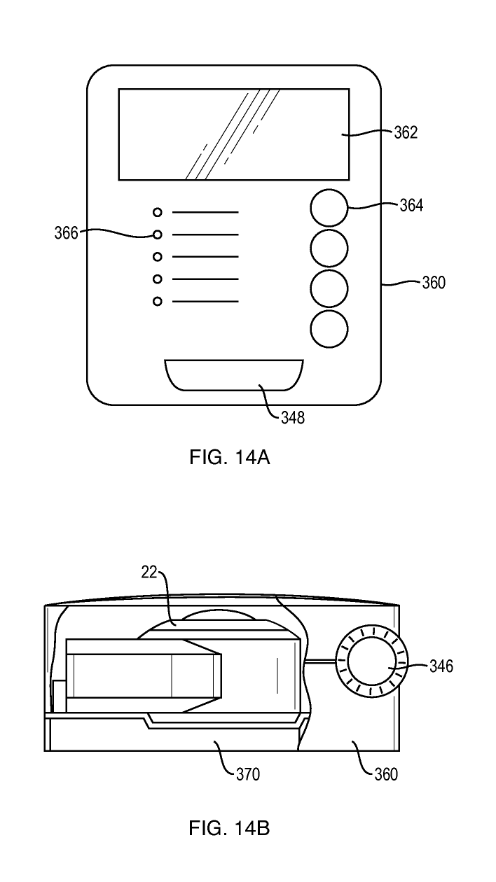

Referring to FIGS. 14A-14B, a top view and a side view of an alternative docking station 360 are shown. The top view shows a large on/off button 348 similar to the docking station 340 shown in FIGS. 11-13. In addition, the docking station 360 has a display 362, such as a color LCD, for displaying information such as mode, pressure, RPM of the motor. In addition, the top of the docking station 360 has a plurality of additional switches 364 and indicator lights 366 for displaying additional information.

Referring to FIG. 14B, the side view of the alternative docking station 360 has a portion broken away. The docking station 360 in addition to being capable of being plugged into an electrical outlet, has a battery pack 370 that provides a back-up power source to the flow generator 22 that overlies the battery pack 370.

It is contemplated in certain models of the flow generator 22, that the flow generator 22 includes an internal power source.

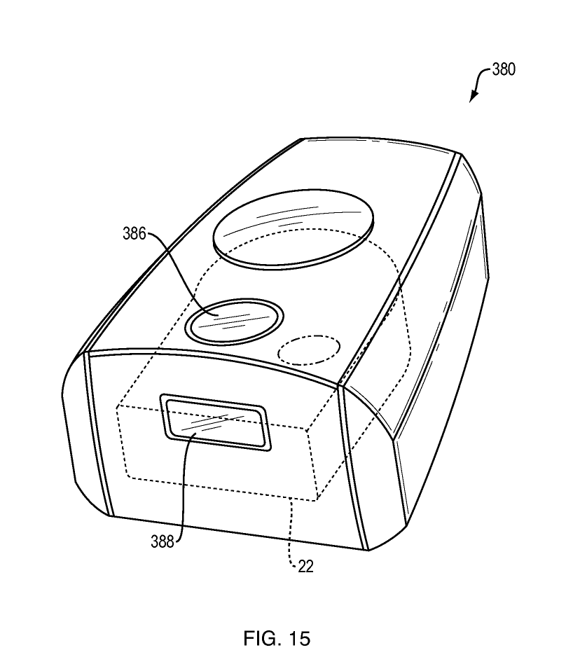

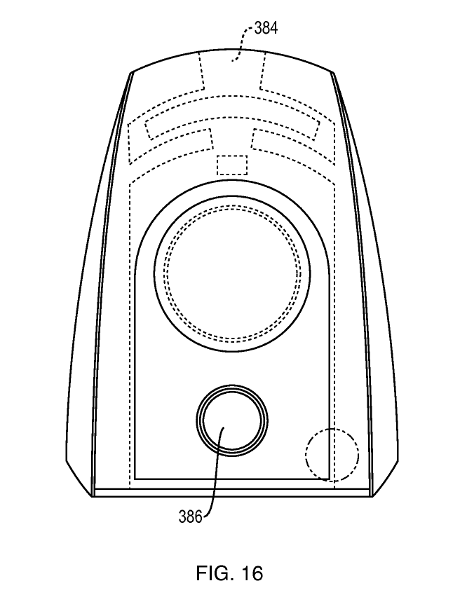

Referring to FIG. 15, a perspective view of a pouch 380 for accepting the blower or flow generator unit 22 is shown. The pouch 380 has several purposes including providing additional acoustic damping 38, as seen in FIG. 16, and providing padding therein allowing the flow generator 22 to be placed in locations such as strapped to the chest as seen in FIG. 7 or located in the bed and the user is able to make contact with the pouch 380 and not having to rest against the hard material of the flow generator 22.

Referring to FIG. 16, a top view of the pouch 380 for accepting the flow generator 22 is shown. Similar to the docking station 340, the pouch 380 has a series of baffles 384 to quiet the device. The pouch 380 has a large opening to allow the user to gain access to the button 178 on the flow generator 22. The pouch 380 has an opening 388 through which hose 26 passes.

As indicated above, the system has mechanisms for sharing and transferring data. Referring back to FIG. 1, the system has various interface devices 48 including removable media 72, a USB port 74, and the blue tooth interface. Referring to FIG. 4, the USB port 74 and a micro SD card reader 72 are shown. Through the interface the user can share data related to the device and the clinician can adjust parameters.

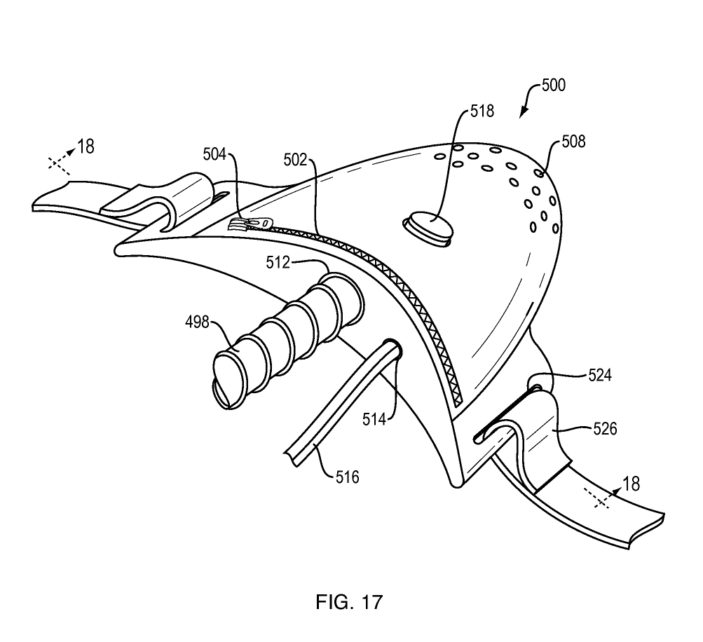

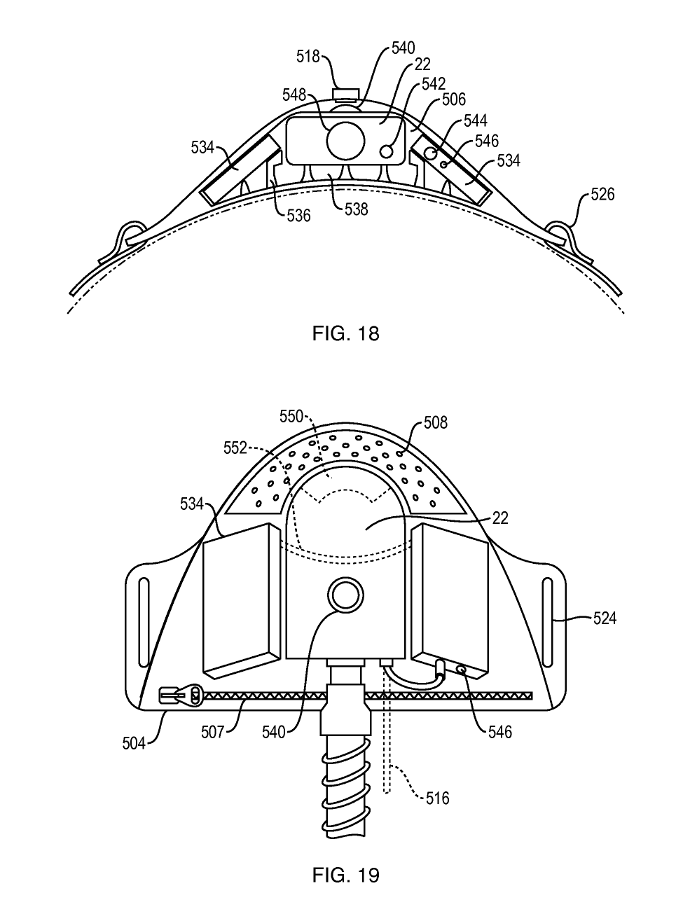

Referring to FIG. 17, a perspective view of an alternative pouch 500 is shown. The pouch 500 has an opening 502 which is closeable such as a zipper 504. The opening 502 grants access to a cavity 506, as seen in FIG. 18. The pouch 500 has a plurality of openings 508 through which air is drawn to provide air to the flow generator 22.

The pouch 500 has a hose opening 512 through which a hose 498 passes to a mask 24. The pouch 500 has a power opening 514 through which a power cord 516 passes. The pouch 500 has a button 518 that overlies the operation button on the flow generator 22. The pouch 500 has a pair of slots 524 for receiving straps 526.

Referring to FIG. 18, a sectional view of the pouch 500 taken along the line 18-18 in FIG. 17 is shown. The cavity 506 that receives the flow generator 22 and the pair of batteries 534 has a series of walls 536 that create passages 538 through which the air passes prior to being drawn into the flow generator 22.

The button 518 on the pouch 500 overlies an operation button 540 on the flow generator 22. The button 518 transmits the user's input to the flow generator 22.

The flow generator has a power receptacle 542. One of the batteries 534 is shown with both a power out port 544 and a power in port 546. The interface 548 on the flow generator 22 is also seen.

Referring to FIG. 19, a top view of an alternative pouch 500 is shown. The opening 502 grants access to a cavity 506, as seen in FIG. 18. The flow generator 22 and the pair of batteries 534 are shown. The flow generator 22 has a filter 550 through which air is drawn; the filter is shown in hidden line. A power cable 552 extending between the two batteries 534 is shown in hidden line. A second power cable 554 extends from the power out port 544 on the battery 534 and the power port 542 of the flow generator.

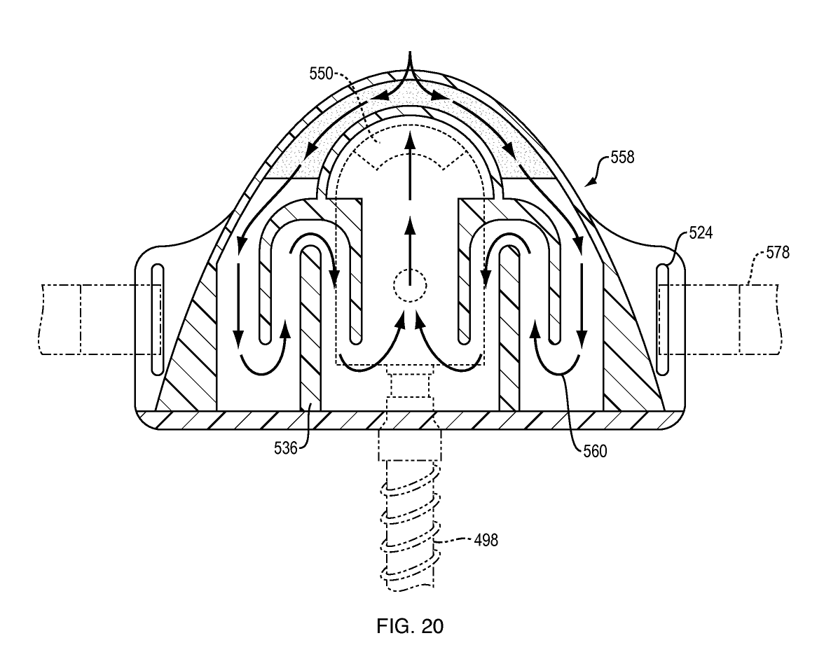

FIG. 20 is a sectional top view of the pouch 500 showing the flow path 560 of the air through the pouch prior to being drawn into the flow generator 22. The series of walls 536 that define passages 538 for the air define an acoustic suppression chamber 558. When the flow generator 22 is operating it emanates acoustic energy. The baffle walls 536 are formed out of an absorbed acoustic foam material which constitutes the acoustic chamber 558. The convoluted path of the acoustic chamber 558 is disposed in a way to optimally absorb acoustic energy.

In one embodiment, the acoustic chamber 558 can be constructed of a more solid material such as high durometer plastic such as PVC or similar material. There may also be a combination of a softer material such as foam and harder material.

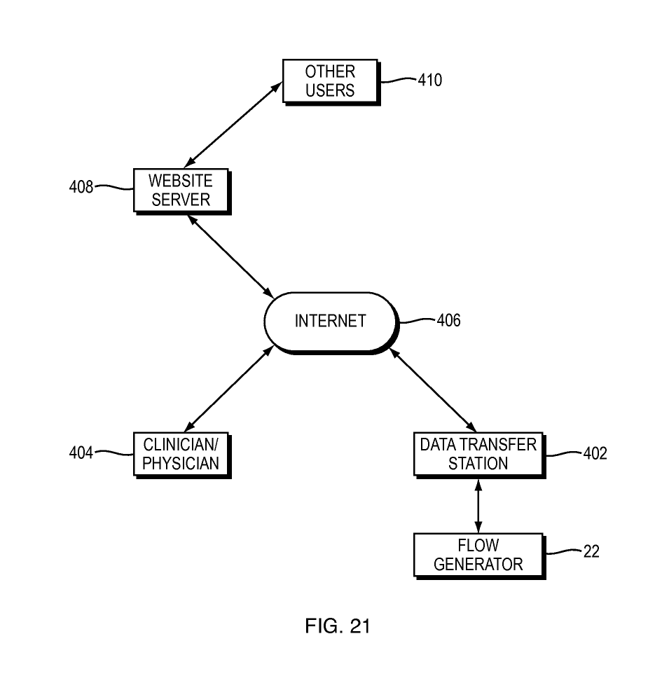

Referring to FIG. 21, a schematic of communication between the flow generator and other devices is shown. The flow generator 22 is connected to a data transfer station 402 via one of various methods such as blue tooth, a USB data cable, or physically transferring a data card from the flow generator to the data transfer station. The data transfer station can be connected to various other persons including a clinician and/or physician 404 via the internet 406. In addition, the flow generator 22 of the system 20 can be connected to a website server 408 that contains data. It is recognized that data can be transferred by other mechanisms such as a direct phone line connection.

The user can allow their personal medical device data that is stored in encrypted form in cloud storage or a web server 408 database to be shared with other users on a granular basis. For instance, Adrian, a user, could be on a message board hosted within the system and elect to share certain data with other users 410 of the message board. Fundamentally, it is a very granular permissions system that allows Adrian to share only what he wants to share in a very granular way. This type of sharing mechanism is used for sharing between users/patients in a social media manner.

In an embodiment, on account creation a user is given a randomly generated alias (or given the ability to create their own alias name) that can be used to decouple the user's data from the user themself. This process would be useful in being able to offer easy syntax for third parties-like clinicians and researchers—to have a ‘John Doe’ like reference to de-identified data and could act as a relational primary key in the database between Adrian's full, identifiable medical data and the subset of data that Adrian has chosen to share on a de-identified basis.

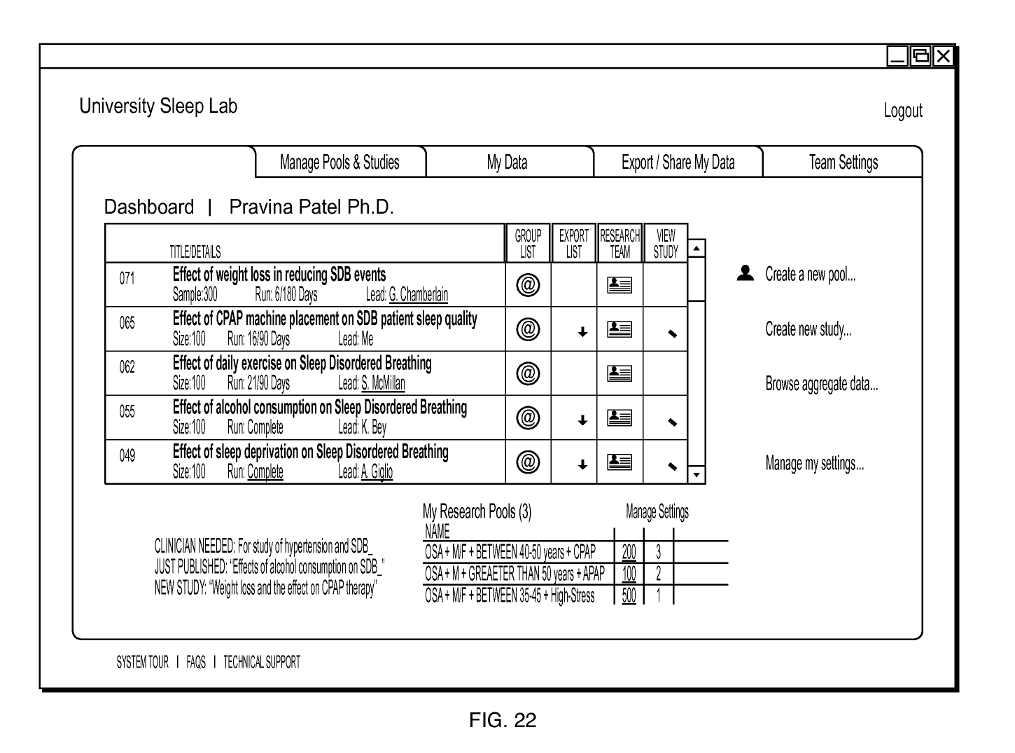

The system has the ability to aggregate data whereby many medical device users are volunteering access to their de-identified device data. By having a system for decoupling data from users' real identities, a clinician, doctor, or research hospital can browse user profiles for the type of patient they are looking for on a very granular level and then invite that user to be a participant in a research study. FIG. 22 shows a screenshot of an example of a clinical dashboard.

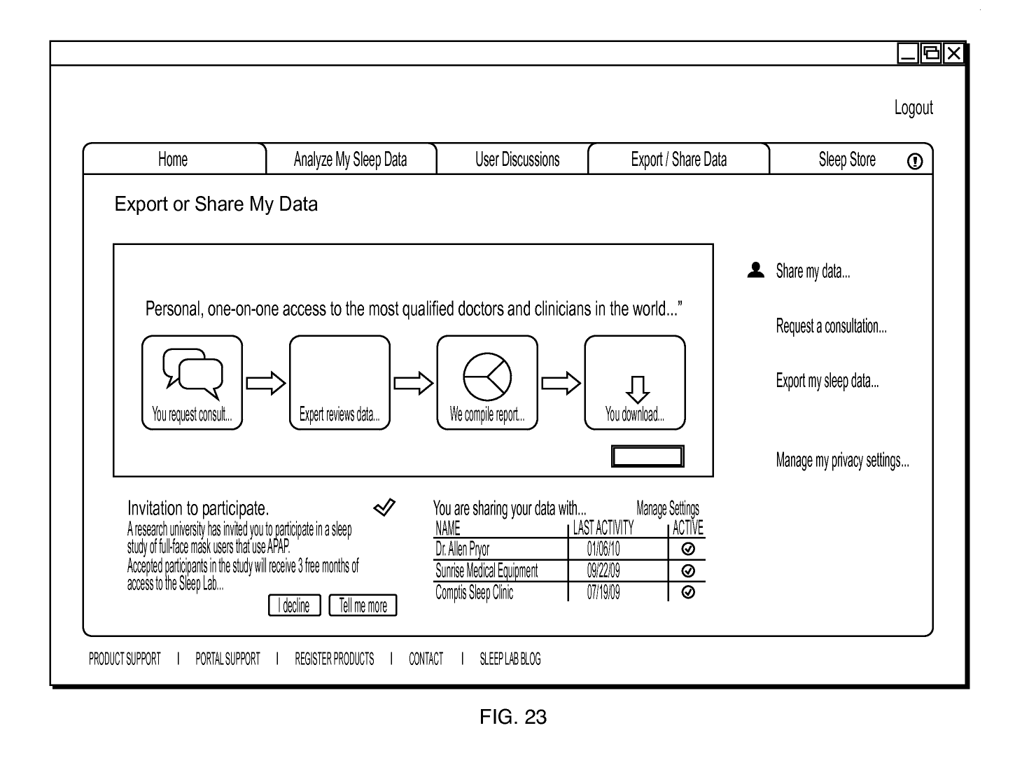

In addition, in certain embodiments the user can request a second opinion from a remotely located doctor, clinician, or specialist with only a few clicks. The user's data is aggregated within the system. By selecting an available provider and granting them permission to view the user's data, the user can get a second opinion from a participating doctor or sleep professional and have their data immediately available to that third party. FIG. 23 shows a second opinion screenshot.

While the principles of the invention have been described herein, it is to be understood by those skilled in the art that this description is made only by way of example and not as a limitation as to the scope of the invention. Other embodiments are contemplated within the scope of the present invention in addition to the exemplary embodiments shown and described herein. Modifications and substitutions by one of ordinary skill in the art are considered to be within the scope of the present invention.

In an embodiment, the orientation sensor 222 or a separate accelerator is used to monitor the motion of the flow generator unit. If the system determines that the compressor is realizing too much external force, the system shuts the compressor down to minimize damage to the impeller. The system then can restart and run diagnostics to determine that the compressor and the system are running properly. The system can adjust the settings and/or generate an error code to inform the user and the clinician.

In addition, the system 20 can have a continuous self-diagnostic that runs upon power being applied to the printed circuit board. In addition, the system can run diagnostics as the system is running.

It is recognized that an additional filter can be placed between the impeller and the cavity 110 of the mask 24.

While the impeller of the compressor of the flow generator is described as rotating counterclockwise, it is recognized that the compressor could be configured to rotate in the other direction.

While the flow generator 22 is described as attached to the body of the user, such as affixed to the user's chest and to the mask, it is recognized that the flow generator 22 could be secured to other locations such as the arm.

While two distinct pressure switches have been described with respect to FIG. 2B, it recognized that a single pressure switch could both monitor sensing treatment pressure and also simultaneously monitor the pneumatic pump for a distinct pressure signal.

What is claimed:

1. An apparatus for delivering pressurized gas to the airway of a patient, the apparatus comprising:
a gas flow generator for providing a flow of gas;
a mask for delivery of gas flow to an airway of a patient;
a connector between the gas flow generator and the mask for providing a flow of gas; and
a mechanism for turning the flow of gas on and off distinct from turning on the apparatus.

2. An apparatus of claim 1 further comprising an orientation sensor wherein the orientation sensor can influence when the flow of pressurized air is turned on and off.

3. A mask for delivery of gas flow to an airway of a patient comprising:
a shell having a rim defining a cavity adapted for interface with a user's nose and mouth, the shell having a connection aperture;
a mask connector interfacing with the connection aperture of the shell, the connector defining a conduit for flow of pressurized air from a flow generator;
an exhaust port being continuously open and having suitable flow resistance for maintaining a pressure in the cavity;
a breathing port adaptable to open when there is no flow of pressurized air for allowing free breathing by the user.

4. A mask of claim 3 further comprising a heat moisture exchange (HME) carried by the mask connector, the HME collecting moisture on exhaling and providing moisture to the air on inhaling.

5. A mask of claim 3 further comprising a port defining a confined space, the port adapted to connect a sensor carried on the flow generator, a flexible membrane covering the port adapted to change the volume of the confined space therein influencing the sensor.

6. A mask of claim 3 further comprising a port adapted to connect to a sensor carried by the flow generator unit for controlling the air flow.

7. An enclosure for a flow generator of a continuous positive airway pressure (CPAP) system, the enclosure comprising:
a housing having an insertion cavity adapted to receive the flow generator; and
the housing defining an input air flow path having a breathable gas outlet for communicating air to an inlet on the flow generator, the flow path including an acoustic chamber for reducing noises.

8. An enclosure of claim 7 wherein the enclosure is a pouch having a pliable material and a closure device for closing the insertion cavity.

9. An enclosure of claim 8 wherein the closure device is a zipper.

10. An enclosure of claim 8 wherein the acoustic chamber has baffle walls with acoustic absorbing foam material.
